(19) United States
(12) Patent Application Publication
(10) Pub. No.: US 2016/0299543 A1
(43) Pub. Date: Oct. 13, 2016

(54) THERMAL MANAGEMENT DEVICE SYSTEMS

(71) Applicant: VORBECK MATERIALS, Jessup, MD (US)

(72) Inventors: LOUISE BROOKS, WASHINGTON, DC (US); JOHN S LETTOW, WASHINGTON, DC (US)

(73) Assignee: VORBECK MATERIALS CORP., JESSUP, MD (US)

(21) Appl. No.: 15/032,414

(22) PCT Filed: Feb. 27, 2014
(86) PCT No.: PCT/US14/19172

§ 371 Date: Apr. 27, 2016

Related U.S. Application Data
(60) Provisional application No. 61/770,310, filed on Feb. 27, 2013.

(51) Int. Cl. G06F 1/20; A61F 7/02; A61F 7/08; A61F 7/10; A41D 13/005; A61F 7/00

(52) U.S. Cl. CPC G06F 1/20; A41D 13/005; A61F 7/007; A61F 7/08; A61F 7/10; A61F 7/02

(57) ABSTRACT

Thermal management device systems comprising a thermal management device component and a computing device component, wherein the thermal management device component comprises compositions that comprise graphene sheets.

### REFERENCE TO RELATED APPLICATIONS

The present application claims priority to U.S. Provisional Application 61/770,310, filed on Feb. 27, 2014, the entire contents of which are hereby incorporated by reference.

### FIELD OF THE INVENTION

The present invention relates to thermal management device systems comprising a thermal management device component and a computing device component, wherein the thermal management device component comprises compositions that comprise graphene sheets.

### SUMMARY OF THE INVENTION

Disclosed and claimed herein is a thermal management device system comprising a thermal management device component and a computing device component, wherein the thermal management device component comprises compositions that comprise graphene sheets. Also disclosed and claimed herein are a thermal management device system comprising a thermal management device component and a computing device component, wherein the thermal management device component comprises at least one heating and/or cooling element that comprises compositions that comprise graphene sheets and a thermal management device system comprising a thermal management device component and a computing device component, wherein the thermal management device component comprises at least one heating and/or cooling element that comprises a coating comprising at least one electrically conductive component.

### BRIEF DESCRIPTION OF THE DRAWINGS

FIG. 1 shows a heating or cooling element comprising graphene sheets.

FIG. 2 shows a heating or cooling element overcoated with a composition comprising graphene sheets.

FIG. 3 shows a printed heating or cooling element overcoated with a composition comprising graphene sheets.

FIG. 4A is a schematic drawing of a data transmission computer connected to a TMD that can transfer data in both directions between the computer and TMD where the TMD has an external power source.

FIG. 4B is a schematic drawing of a data transmission computer connected to a TMD where the data transmission computer provides power to the TMD.

FIG. 4C is a schematic drawing of a data transmission computer connected to a TMD where the data transmission computer provides power to the TMD via a controller that is part of the data transmission computer.

FIG. 4D is a schematic drawing of a data transmission computer connected to a TMD where the TMD has an external power source that is controlled by the data transmission computer controller.

FIG. 4E is a schematic drawing of a data transmission computer having a power source that connected to a TMD where the TMD is powered by an external power source and the data transmission computer power source and where both power sources are controlled by the data transmission computer. The data transmission computer can optionally also provide power to the power source.

FIG. 4F is a schematic drawing of a data transmission computer connected to a TMD where the data transmission computer provides power to the TMD via a H/CE that is incorporated into the TMD.

FIG. 5 shows a thermal management device in the form of a heated bandage that is connected to a computer.

FIG. 6 shows a composition comprising graphene sheets that is overcoated with heating or cooling elements.

FIG. 7 shows a composition comprising graphene sheets that is overcoated with heating or cooling elements.

### DETAILED DESCRIPTION OF THE INVENTION

The thermal management device system comprises a thermal management device (referred to as “TMD”) component connected to one or more computing devices (“computer”) components in such a way that allows data transmission from one or more TMDs to the computer(s) and/or from the computer(s) to the TMDs. The computer components are referred to as “data transmission computers” (also referred to also as the “DTC”). The TMD has at least one heating and/or cooling element (“H/CE”). Examples of H/CEs include heating elements (including resistance heating elements) and cooling elements (such as thermo-electric coolers).

By thermal management device is meant a device or component of a device that is designed or used to generate, spread, disperse, otherwise handle or manage the flow of heat. Examples include heaters, heating elements, heat sinks, thermal diffusion devices, heat spreaders, coolers, thermally conductive adhesives, thermally conductive gaskets and seals. etc.

A computer may refer to any number of types of devices with processing power, such as a personal computer (desktop or laptop), a “smart phone” (such as an iPhone, Android phone, etc.), a tablet computer (such as an iPad, Kindle, etc.), a personal digital assistant, a custom PCB board etc. The computer can comprise components such as display or other output device, data transmission ports and hardware, power supplies, etc. The computer can also function as a controller.

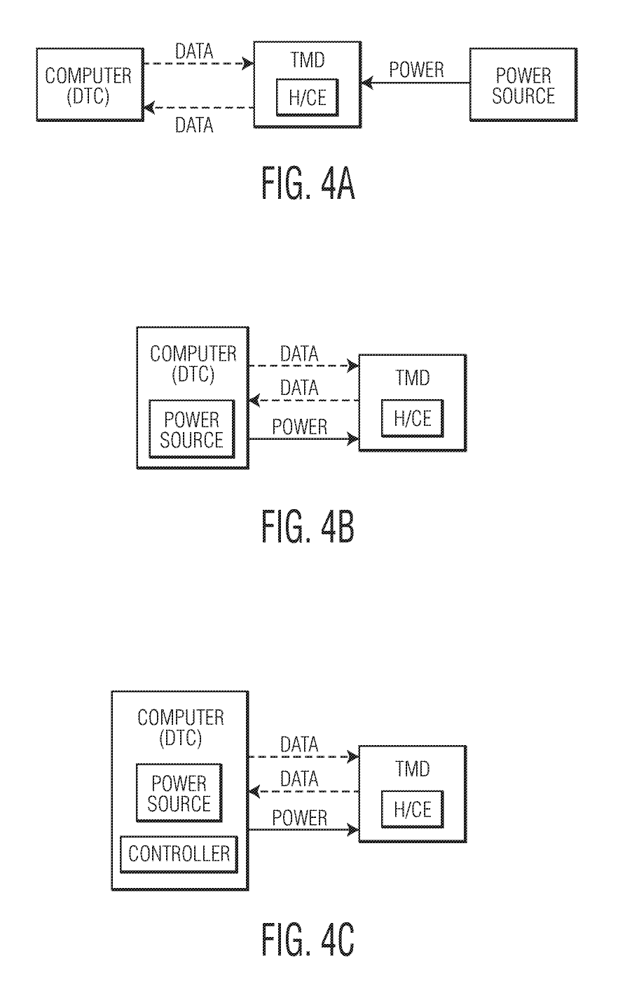

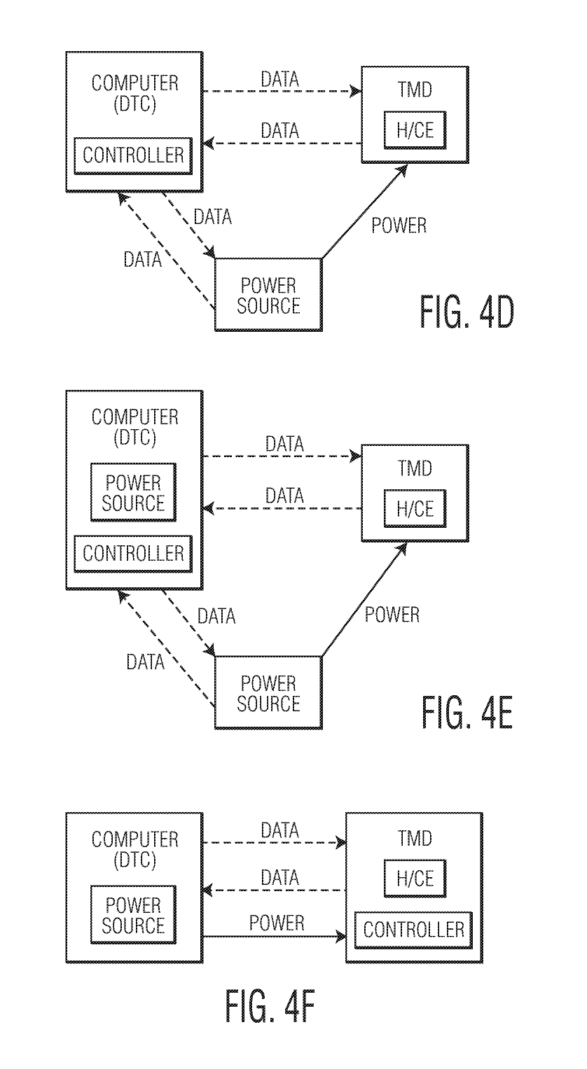

FIG. 4A shows a data transmission computer connected to a TMD that can transfer data in both directions between the computer and TMD where the TMD has an external power source.

FIG. 4B shows a data transmission computer connected to a TMD where the data transmission computer provides power to the TMD.

One or more power sources can be used to supply power (such as electrical power) to the TMD. The TMD can have its power source be a computer having a power source component in whole or part. A computer providing power to the TMD can be the same as or different from the DTC. The TMD can have a power source that is separate from the DTC or in addition to the DTC. Power sources that are separate from the DTC can in some cases be controlled by the DTC.

The power source may be an integral part of a device such as a computer. The power source may also be an integral part of the TMD (such as batteries). The power source may also be an external device, such as a battery, wall plug, automotive power source, etc. A single power source may be utilized or several power sources may be used in conjunction with each other, for example: a wall outlet and a battery. The power source may supply alternating current or direct current. The power source may be modulated, for example, in order to vary the amount of power being dissipated by a heating element in the TMD. Examples of modulation include pulse width modulation. The power source can be a low voltage power source such a USB port, 12 V power supplies and batteries, 24 and 42 V batteries and power supplies, cell phone batteries, 9 V batteries, AAA batteries, AA batteries, coin cells, etc. Laptop computer batteries, car batteries, etc. can be used as power sources. Power may be supplied to the device using, for example, a USB connection from a computer (such as laptop computers), from a wall plug or other (e.g. a 12 V car battery) USB power adapter, etc. Power may be transmitted from DTC to the TMD using the same method as data transmission, such as by using a USB connection (for example, by running a cable between USB ports on the DTC and TMD).

In some cases, the power source can be controlled by one or more controllers. The controllers can be used to control the temperature output (e.g. heat or cooling temperatures) of the TMD. It may be manually controlled by the user to achieve a desired power output. It may also be used in conjunction with one or more temperature sensors and a feedback control loop to maintain a constant temperature. It may be programmed to turn on or off after a specified amount of time has passed or to cycle on and off according to a schedule. The controls may be input by the user, pre-programmed into the TMD, provided by the DTC, adjusted remotely via an external connection such as the Internet, etc.

The controllers can be incorporated into the DTC and/or the TMD, or can be external to both.

FIG. 4C is a schematic drawing of a data transmission computer connected to a TMD where the data transmission computer provides power to the TMD via a controller that is part of the data transmission computer.

FIG. 4D is a schematic drawing of a data transmission computer connected to a TMD where the TMD has an external power source that is controlled by the data transmission computer controller.

FIG. 4E is a schematic drawing of a data transmission computer having a power source that connected to a TMD where the TMD is powered by an external power source and the data transmission computer power source and where both power sources are controlled by the data transmission computer. The data transmission computer can optionally also provide power to the power source.

FIG. 4F is a schematic drawing of a data transmission computer connected to a TMD where the data transmission computer provides power to the TMD via a controller that is incorporated into the TMD.

Data can be transferred between the DTC and TMD (or other components, such as an external power source) using any suitable interface method, such as wired or wireless connections, including USB, serial, parallel, HDMI, Firewire, SCSI, ethernet, Wi-Fi, Bluetooth, infrared, etc. connections. Data may be transferred between devices in both directions or just one direction.

Data transferred from the DTC to the TMD and displayed or otherwise output on a display or other output device incorporated into the TMD or external to the TMD. The display may be any device for displaying graphics or information such as a computer monitor, a laptop screen, a tablet screen, a “smart phone” screen, an electrophoretic ink display, an LCD screen, an LED array, etc. Data transferred from the TMD to the DTC can be displayed or otherwise output to a display or other output device incorporated into the DTC or external to the DTC. Additionally, data may be transmitted to the users in the form of sound, vibration, etc. via other output devices in addition to or instead of the display.

The TMD can be controlled via software on the DTC. The software may be used for many purposes, such as to adjust temperatures, set temperature profiles, vary temperatures over time, etc. Software can be used to monitor data taken from the TMD and send data back to the TMD for display. Software may be used to log data (such as temperature and power) over time.

The thermal management system may convey a variety of information. Information may be conveyed to the user, such as the power output, current temperature, temperature setpoint, control scheme, device information, instructions, operating history, etc. Additionally the system may convey advertisements, especially advertisements that would be relevant to a user of the thermal management device. Information can be downloaded from or uploaded to the internet. The TMD may be configured such that when it is connected to the DTC, information (such as an advertisement), which can in some cases be downloaded from the internet, is automatically displayed on the DTC or TMD. Information may also be conveyed to one or more third parties about the use of the thermal management device.

The thermal management device may be used to heat or cool some portion of the body. Heating or cooling may be desired for a variety of purposes, such as medical treatment, therapy, rehabilitation, comfort, thermal conditioning, bio-feedback, etc. The thermal management device may be applied to any part of the body or combination of parts, such as joints, muscles, extremities, head, abdomen, skin, etc.

Examples of types of thermal management devices include medical and health-related devices, portable heaters and coolers, heaters and coolers for food and beverages (such as coffee, carbonated drinks, alcoholic drinks, etc.), chemical curing devices, etc. Examples of medical devices include heating and cooling pads, bandages (such as ace bandages), splints, braces, casts, etc.

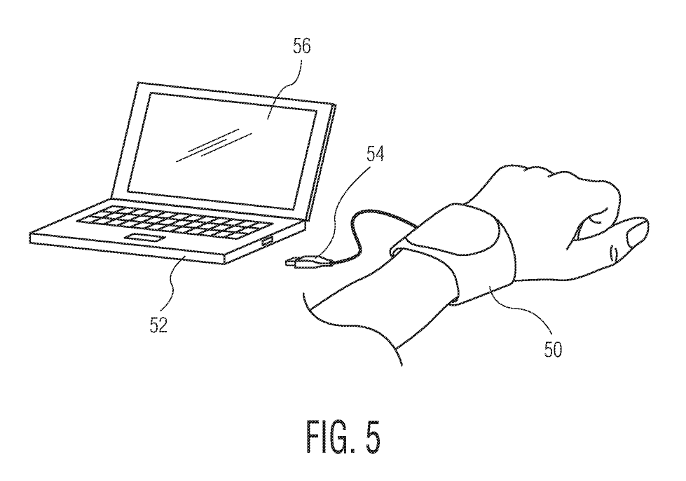

FIG. 5 shows a thermal management device 50 in the form of a heated bandage. The bandage is connected to a laptop computer 52 via a cable and connector 54. Power can be supplied to the bandage from the computer via the cable. The computer has a display screen 56 that can display advertisements, information about the bandage temperature, heating time, etc.

The thermal management devices have components (such as heating and/or cooling elements) that comprise compositions comprising graphene sheets. They may further comprise other components, including other electrically conductive materials. By heating and/or cooling element is meant a device or component of a device that is designed or used to generate, spread, disperse, otherwise handle or manage the flow of heat. Examples include heaters, heating elements, heat sinks, thermal diffusion devices, heat spreaders, coolers, thermally conductive adhesives, thermally conductive gaskets and seals. etc.

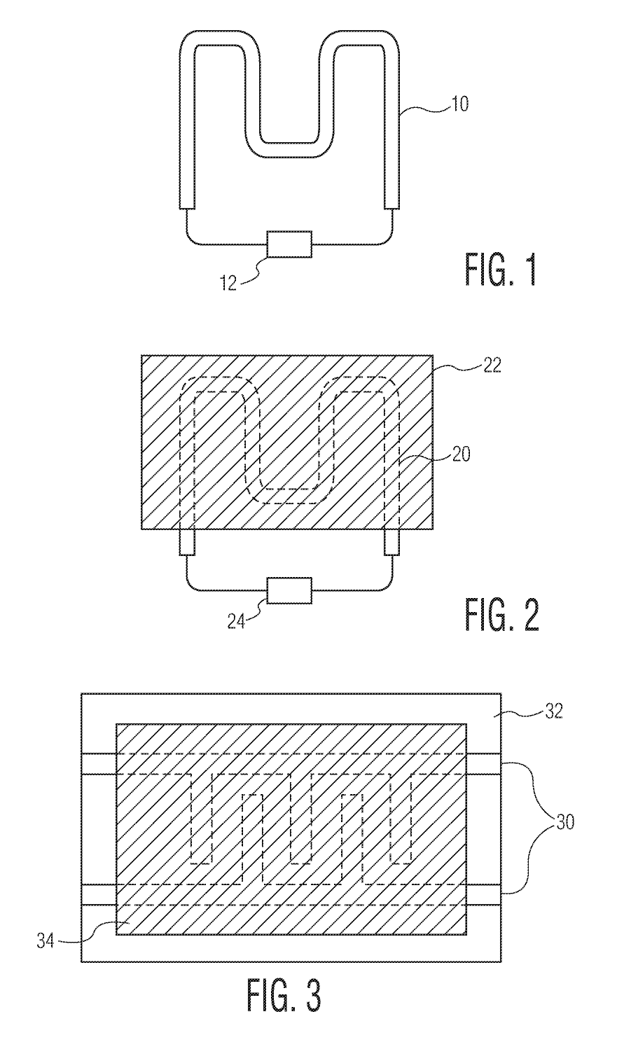

The composition comprising graphene sheets may be used to make a heating or cooling element. For example, FIG. 1 shows a heating or cooling element 10 comprising a composition comprising graphene sheets connected to a power source 12. When a voltage is applied, the element warms up.

In a thermal management device, the compositions may be used to embed or cover all or a part of a heating or cooling element, serving as a heat spreader. The heating or cooling element can be made from a composition comprising graphene sheets and/or other materials. FIG. 2 shows a heating or cooling element 20 covered with a coating 22 comprising graphene sheets. The in the case of a heating element, the element can be connected to a voltage source 24. In the case of a cooling element, the element can be in contact with or proximity to a heat source. The coating 22 can act as a heat spreader or diffuser.

The graphene sheets composition may further comprise one or more additional components. Examples of other materials include polymers, other thermally and/or electrically conductive materials, etc. The composition can be a polymer composite, a coating or ink, or the like.

The heating and/or cooling elements can be formed from the compositions using any suitable method. For example, they may be molded, extruded, or the like or applied in the form of a coating. The compositions can be overmolded or coated onto a heat source, heating element, cooling element, etc. A heat or cooling source or element can be partially or fully embedded into the compositions.

The devices can be formed by creating an electrically conductive heating or cooling element from a metallic (such as silver, copper, aluminum, steel, etc.) or non-metallic material and overcoating or overmolding the heating or cooling element with a form of the graphene sheets composition that is more electrically resistive than the heating or cooling element. Alternatively, the graphene sheets composition can be coated, molded, etc. over the more electrically conductive material. The composition can act as a heat spreader. The heating or cooling element can also be formed from a graphene sheet composition. The heating or cooling element can be a wire or filament, a trace, a printed trace, a metallized or plated surface, a metallic adhesive, etched, etc. The heating or cooling elements can be connected to a voltage source and act as bus bars.

FIG. 3 shows heating or cooling elements 30 printed on a surface 32 and overcoated with graphene sheets composition 34. Overcoating 34 can heat up as a current is applied to the heating elements and can serves as a heat spreader or diffuser.

The more electrically conductive materials can be made of any suitable electrically conductive material. They can be metals or metal alloys (e.g. copper, aluminum, silver, gold, etc.), organic, polymeric, and/or carbon-based conductors etc., coatings or inks, etc. Conductive material can be in any suitable form, including strips, sheets, foils, tapes, wires, tapes, threads, etc. Conductive materials can be deposited, such as by sputtering, plating, etching, molding, printing, coating, metallization, vapor deposition, adhering, gluing, taping, or other deposition techniques.

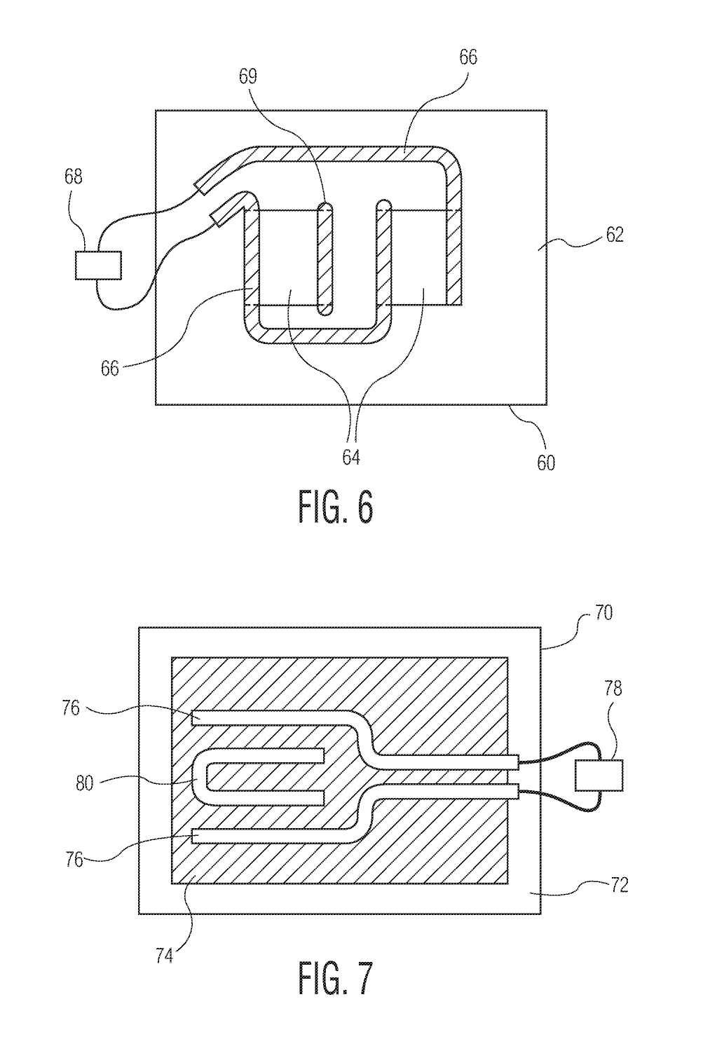

FIG. 6 shows a thermal management device 60 comprising a surface 62 that is coated with graphene sheets composition 64. The graphene sheets composition is overcoated with heating or cooling elements/bus bars 66, which are connected to a voltage source 68, and heating or cooling element 69, which is not connected to a voltage source.

FIG. 7 shows a thermal management device 70 comprising a surface 72 that is coated with graphene sheets composition 74. The graphene sheets composition is overcoated with heating or cooling elements/bus bars 76, which are connected to a voltage source 78, and heating or cooling element 80, which is not connected to a voltage source.

In some embodiments, the graphene sheets composition is applied to a substrate and overcoated with an ink or coating (e.g. silver, copper, etc. ink) to form a heating or cooling element. Alternatively, a heating or cooling element can be formed on a substrate from an ink or coating (e.g. silver, copper, etc. ink) and then overcoated with the graphene sheets composition. In some cases, some parts of the substrate may be coated with the graphene sheets composition and overcoated with the element, while other parts of the substrate are coated with the element and overcoated with the graphene sheets composition. The resulting article can be laminated with other materials and substrates and formed into a heating/cooling device. The other materials and substrates can be laminated on top of the coated surface (e.g. elements and graphene sheets composition) or the device can be overcoated to create a sandwich structure. Substrates can include fabrics, textiles, films, sheets, and other flexible substrates. In some cases, the laminated article can be thermally sealed.

The H/CEs and TMDs can be heat sinks, thermally conductive adhesives, thermal traces, heaters, coolers, passive solar heaters (such as hot water heaters), thermostats, thermally conductive channels, etc. Heaters and coolers can be used for portable heaters and coolers (such as those run off batteries), components of buildings, heaters and coolers for tents, heaters and coolers for clothing (such as outerwear), buildings and building components (such as windows, doors, floors, etc.), pipes (such as outdoor water pipes), medical devices, heating pads, heating patches, heaters and window defrosters for vehicles (such as cars, trucks, motorcycles, forklifts, airplanes, farm equipment), packaging, hot or cold food and beverage packaging, etc.

The thermal management device may be used to heat or cool some portion of the body. Heating or cooling may be desired for a variety of purposes, such as medical treatment, therapy, rehabilitation, comfort, thermal conditioning, bio-feedback, etc. The thermal management device may be applied to any part of the body or combination of parts, such as joints, muscles, extremities, head, abdomen, skin, etc.

Examples of types of thermal management devices include medical and health-related devices, portable heaters and coolers, heaters and coolers for food and beverages (such as coffee, carbonated drinks, alcoholic drinks, etc.), chemical curing devices, etc. Examples of medical devices include heating and cooling pads, bandages (such as ace bandages), splints, braces, casts, etc.

The thermal management devices can be flexible and made to conform to surfaces. They can be crease resistant and thin. They can be used in items such as apparel, bags, gear, etc. Examples include shirts, jackets, coats, vests, shirts, pants, shorts, hats, helmets, shoes, boots, belts, gloves and mittens, socks, underwear, sweat shirts and pants, athletic apparel and gear, hand bags, purses, backpacks, briefcases, messenger bags, computer bags, satchels, luggage, sports bags (golf bags, gym bags, etc.), tents, sleeping bags, sleeping pads and mattresses, hunting and sports equipment, ski apparel (such as ski jackets, pants, boots, etc.) chairs, cushions, upholstered objects, seats (such as car or vehicle seats), ballistic protection equipment (e.g. bullet-proof vests), scuba diving equipment, etc. They can be used as pocket warmers.

Devices (such as heaters) can be incorporated into or onto the items by any suitable method, such as by sewing, snaps, buttons, tape, adhesive, hook and loop fasteners (e.g. Velcro®), zippers, etc. They can be embedded into the object either permanently or removable. They can be placed in pockets, slits, hems, between layers of components of the objects, etc. The can be placed within the padding of bags such as backpacks, computer bags, messenger bags, etc. They can be sewn, taped, zipped, laminated, etc. into place. They can be washable in some embodiments. Devices can be printed onto fabrics that can be incorporated into apparel or other gear. The fabrics can be laminated with other fabric materials.

The devices can be used with alternating and direct current power sources, such as energy from an electrical grid, batteries (rechargeable and non-rechargeable), solar power, fuel cells, capacitors, solar power, etc. The power source can be a low voltage power source such a USB port, 12 V power supplies and batteries, 24 and 42 V batteries and power supplies, cell phone batteries, 9 V batteries, AAA batteries, AA batteries, coin cells, etc. Laptop computer batteries, car batteries, etc. can be used as power sources. Power may be supplied to the device using, for example, a USB connection from a computer (such as laptop computers), from a wall plug or other source (e.g. a 12 V car battery) USB power adapter, etc.

In some cases, the devices can have anisotropic thermal and/or electrical conductivities.

In one embodiment, a metallic heating element is formed on a heat sealable substrate by any suitable method, such as printing, metal deposition, using a conductive adhesive, applying cut (such as die-cut) shapes, etc. The heating element is overcoated at least in part with a graphene sheets coating composition, where the coating composition is less thermally conductive than the heating element material. The heat sealable substrate containing the overcoated heating element can then adhered to one or more layers of other materials, such as fabrics. It can be directly adhered to an article of apparel or other item.

Examples of electrically and/or thermally conductive additives include metals (including metal alloys), conductive metal oxides, conductive carbons, polymers, metal-coated materials, inorganic compounds, ceramics, etc. These components can take a variety of forms, including particles, powders, flakes, foils, needles, etc.

Metals can be pure metals, alloys, etc. Examples of metals include, but are not limited to silver, copper, aluminum, platinum, palladium, nickel, chromium, gold, zinc, tin, iron, gold, lead, steel, stainless steel, rhodium, titanium, tungsten, magnesium, brass, bronze, colloidal metals, etc. Examples of metal oxides include antimony tin oxide and indium tin oxide and materials such as fillers coated with metal oxides. Metal and metal-oxide coated materials include, but are not limited to metal coated carbon and graphite fibers, metal coated glass fibers, metal coated glass beads, metal coated ceramic materials (such as beads), etc. These materials can be coated with a variety of metals, including nickel.

Examples of conductive polymers include, but are not limited to, polyacetylene, polyethylene dioxythiophene (PEDOT), poly(styrenesulfonate) (PSS), PEDOT:PSS copolymers, polythiophene and polythiophenes, poly(3-alkylthiophenes), poly(2,5-bis(3-tetradecylthiophen-2-yl)thieno[3,2-b]thiophene) (PBTTT), poly(phenylenevinylene), polypyrene, polycarbazole, polyazulene, polyazepine, polyflurorenes, polynaphthalene, polyisonaphthalene, polyaniline, polypyrrole, poly(phenylene sulfide), polycarbozoles, polyindoles, polyphenylenes, copolymers of one or more of the foregoing, etc., and their derivatives and copolymers. The conductive polymers can be doped or undoped. They can be doped with boron, phosphorus, iodine, etc.

Examples of conductive carbons include, but are not limited to, graphite (including natural, Kish, and synthetic, annealed, pyrolytic, highly oriented pyrolytic, etc. graphites), graphitized carbon, carbon black, carbon fibers and fibrils, carbon whiskers, vapor-grown carbon nanofibers, metal coated carbon fibers, carbon nanotubes (including single- and multi-walled nanotubes), fullerenes, activated carbon, carbon fibers, expanded graphite, expandable graphite, graphite oxide, hollow carbon spheres, carbon foams, etc.

Thermally conductive additives can be dielectrics. Thermally conductive additives can be metal based. They can be electrically conducting, insulating or semiconducting. In some embodiments, the additives have electrical conductivities of no more than about 10⁵ S/cm, or of no more than about 10⁴ S/cm, or of no more than about 10³ S/cm, or of no more than about 10² S/cm, or of no more than about 10 S/cm, or of no more than about 1 S/cm, or of no more than about 0.1 S/cm, or of no more than about 10⁻² S/cm, or of no more than about 10⁻³ S/cm, or of no more than about 10⁻⁵ S/cm, or of no more than about 10⁻⁷ S/cm, or of no more than about 10⁻⁸ S/cm, or of no more than about 10⁻⁹ S/cm.

Examples of thermally conductive additives include metal oxides, nitrides, ceramics, minerals, silicates, etc. Examples include boron nitride, aluminum nitride, alumina, aluminum nitride, berylium oxide, nickel oxide, titanium dioxide, copper(I) oxide, copper (II) oxide, iron(II) oxide, iron(I,II) oxide (magnetite), iron (III) oxide, iron sulfide, iron(II) sulfide, silicon dioxide, zinc oxide, magnesium oxide (MgO), etc.

In some embodiments, additives have a thermally conductivity at 25° C. of at least about 0.5 W/m·K, of at least about 0.7 W/m·K, of at least about 1 W/m·K, or at least about 3 W/m·K, or at least about 5 W/m·K, or at least about 10 W/m·K, or at least about 20 W/m·K, or at least about 30 W/m·K.

The compositions comprising graphene sheets can comprise graphene sheets and at least one inorganic thermally conductive additive that is non-electrically conductive. The non-electrically conductive additives can be metal based. In some embodiments, they have a thermally conductivity at 25° C. of at least about 0.5 W/m·K, of at least about 0.7 W/m·K, of at least about 1 W/m·K, or at least about 3 W/m·K, or at least about 5 W/m·K, or at least about 10 W/m·K, or at least about 20 W/m·K, or at least about 30 W/m·K.

The non-electrically conductive additive can be electrically insulating or semiconducting. In some embodiments, the additives have electrical conductivities of no more than about 10⁵ S/cm, or of no more than about 10⁴ S/cm, or of no more than about 10³ S/cm, or of no more than about 10² S/cm, or of no more than about 10 S/cm, or of no more than about 1 S/cm, or of no more than about 0.1 S/cm, or of no more than about 10⁻² S/cm, or of no more than about 10⁻³ S/cm, or of no more than about 10⁻⁵ S/cm, or of no more than about 10⁻⁷ S/cm, or of no more than about 10⁻⁸ S/cm, or of no more than about 10⁻⁹ S/cm.

Examples of non-electrically conductive additives include Examples of thermally conductive additives include metal oxides, nitrides, ceramics, minerals, silicates, etc. Examples include boron nitride, aluminum nitride, alumina, aluminum nitride, berylium oxide, nickel oxide, titanium dioxide, copper(I) oxide, copper (II) oxide, iron(II) oxide, iron(I,II) oxide (magnetite), iron (III) oxide, iron sulfide, iron(II) sulfide, silicon dioxide, zinc oxide, magnesium oxide (MgO), etc.

The graphene sheets are graphite sheets preferably having a surface area of from about 100 to about 2630 m²/g. In some embodiments, the graphene sheets primarily, almost completely, or completely comprise fully exfoliated single sheets of graphite (these are approximately ≦1 nm thick and are often referred to as “graphene”), while in other embodiments, at least a portion of the graphene sheets can comprise partially exfoliated graphite sheets, in which two or more sheets of graphite have not been exfoliated from each other. The graphene sheets can comprise mixtures of fully and partially exfoliated graphite sheets. Graphene sheets are distinct from carbon nanotubes. Graphene sheets can have a “platy” (e.g. two-dimensional) structure and do not have the needle-like form of carbon nanotubes. The two longest dimensions of the graphene sheets can each be at least about 10 times greater, or at least about 50 times greater, or at least about 100 times greater, or at least about 1000 times greater, or at least about 5000 times greater, or at least about 10,000 times greater than the shortest dimension (i.e. thickness) of the sheets.

Graphene sheets are distinct from expanded, exfoliated, vermicular, etc. graphite, which has a layered or stacked structure in which the layers are not separated from each other. The graphene sheets do not need to be entirely made up of carbon, but can have heteroatoms incorporated into the lattice or as part of functional groups attached to the lattice. The lattice need not be a perfect hexagonal lattice and may contain defects (including five- and seven-membered rings).

Graphene sheets can be made using any suitable method. For example, they can be obtained from graphite (including natural, Kish, and synthetic, annealed, pyrolytic, highly oriented pyrolytic, etc. graphites), graphite oxide, expandable graphite, expanded graphite, etc. They may be obtained by the physical exfoliation of graphite, by for example, peeling, grinding, milling, graphene sheets. They made be made by sonication of precursors such as graphite. They may be made by opening carbon nanotubes. They may be made from inorganic precursors, such as silicon carbide. They may be made by chemical vapor deposition (such as by reacting a methane and hydrogen on a metal surface). They may be made by epitaxial growth on substrates such as silicon carbide and metal substrates and by growth from metal-carbon melts. They made by made They may be may by the reduction of an alcohol, such ethanol, with a metal (such as an alkali metal like sodium) and the subsequent pyrolysis of the alkoxide product (such a method is reported in Nature Nanotechnology (2009), 4, 30-33). They may be made from small molecule precursors such as carbon dioxide, alcohols (such as ethanol, methanol, etc.), alkoxides (such as ethoxides, methoxides, etc., including sodium, potassium, and other alkoxides). They may be made by the exfoliation of graphite in dispersions or exfoliation of graphite oxide in dispersions and the subsequently reducing the exfoliated graphite oxide. Graphene sheets may be made by the exfoliation of expandable graphite, followed by intercalation, and ultrasonication or other means of separating the intercalated sheets (see, for example, Nature Nanotechnology (2008), 3, 538-542). They may be made by the intercalation of graphite and the subsequent exfoliation of the product in suspension, thermally, etc. Exfoliation processes may be thermal, and include exfoliation by rapid heating, using microwaves, furnaces, hot baths, etc.

Graphene sheets can be made from graphite oxide (also known as graphitic acid or graphene oxide). Graphite can be treated with oxidizing and/or intercalating agents and exfoliated. Graphite can also be treated with intercalating agents and electrochemically oxidized and exfoliated. Graphene sheets can be formed by ultrasonically exfoliating suspensions of graphite and/or graphite oxide in a liquid (which can contain surfactants and/or intercalants). Exfoliated graphite oxide dispersions or suspensions can be subsequently reduced to graphene sheets. Graphene sheets can also be formed by mechanical treatment (such as grinding or milling) to exfoliate graphite or graphite oxide (which would subsequently be reduced to graphene sheets).

Graphene sheets may be made by the reduction of graphite oxide. Reduction of graphite oxide to graphene may be done by thermal reduction/annealing, chemical reduction, etc. and may be carried out on graphite oxide in a solid form, in a dispersion, etc. Examples of useful chemical reducing agents include, but are not limited to, hydrazines (such as hydrazine (in liquid or vapor forms, N,N-dimethylhydrazine, etc.), sodium borohydride, citric acid, hydroquinone, isocyanates (such as phenyl isocyanate), hydrogen, hydrogen plasma, etc. A dispersion or suspension of exfoliated graphite oxide in a carrier (such as water, organic solvents, or a mixture of solvents) can be made using any suitable method (such as ultrasonication and/or mechanical grinding or milling) and reduced to graphene sheets. Reduction can be solvothermal reduction, in solvents such as water, ethanol, etc. This can for example be done in an autoclave at elevated temperatures (such as those above about 200° C.).

Graphite oxide can be produced by any method known in the art, such as by a process that involves oxidation of graphite using one or more chemical oxidizing agents and, optionally, intercalating agents such as sulfuric acid. Examples of oxidizing agents include nitric acid, nitrates (such as sodium and potassium nitrates), perchlorates, potassium chlorate, sodium chlorate, chromic acid, potassium chromate, sodium chromate, potassium dichromate, sodium dichromate, hydrogen peroxide, sodium and potassium permanganates, phosphoric acid (H₃PO₄), phosphorus pentoxide, bisulfites, etc. Preferred oxidants include KClO₄; HNO₃ and KClO₃; KMnO₄ and/or NaMnO₄; KMnO₄ and NaNO₃; K₂S₂O₈ and P₂O₅ and KMnO₄; KMnO₄ and HNO₃; and HNO₃. Preferred intercalation agents include sulfuric acid. Graphite can also be treated with intercalating agents and electrochemically oxidized. Examples of methods of making graphite oxide include those described by Staudenmaier (Ber. Stsch. Chem. Ges. (1898), 31, 1481) and Hummers (J. Am. Chem. Soc. (1958), 80, 1339).

One example of a method for the preparation of graphene sheets is to oxidize graphite to graphite oxide, which is then thermally exfoliated to form graphene sheets (also known as thermally exfoliated graphite oxide), as described in US 2007/0092432, the disclosure of which is hereby incorporated herein by reference. The thusly formed graphene sheets can display little or no signature corresponding to graphite or graphite oxide in their X-ray diffraction pattern.

The thermal exfoliation can be carried out in a continuous, semi-continuous batch, etc. process.

Heating can be done in a batch process or a continuous process and can be done under a variety of atmospheres, including inert and reducing atmospheres (such as nitrogen, argon, and/or hydrogen atmospheres). Heating times can range from under a few seconds or several hours or more, depending on the temperatures used and the characteristics desired in the final thermally exfoliated graphite oxide. Heating can be done in any appropriate vessel, such as a fused silica, mineral, metal, carbon (such as graphite), ceramic, etc. vessel. Heating can be done using a flash lamp or with microwaves. During heating, the graphite oxide can be contained in an essentially constant location in single batch reaction vessel, or can be transported through one or more vessels during the reaction in a continuous or batch mode. Heating can be done using any suitable means, including the use of furnaces and infrared heaters.

Examples of temperatures at which the thermal exfoliation and/or reduction of graphite oxide can be carried out are at least about 150° C., at least about 200° C., at least about 300° C., at least about 400° C., at least about 450° C., at least about 500° C., at least about 600° C., at least about 700° C., at least about 750° C., at least about 800° C., at least about 850° C., at least about 900° C., at least about 950° C., at least about 1000° C., at least about 1100° C., at least about 1500° C., at least about 2000° C., and at least about 2500° C. Preferred ranges include between about 750 about and 3000° C., between about 850 and 2500° C., between about 950 and about 2500° C., between about 950 and about 1500° C., between about 750 about and 3100° C., between about 850 and 2500° C., or between about 950 and about 2500° C.

The time of heating can range from less than a second to many minutes. For example, the time of heating can be less than about 0.5 seconds, less than about 1 second, less than about 5 seconds, less than about 10 seconds, less than about 20 seconds, less than about 30 seconds, or less than about 1 min. The time of heating can be at least about 1 minute, at least about 2 minutes, at least about 5 minutes, at least about 15 minutes, at least about 30 minutes, at least about 45 minutes, at least about 60 minutes, at least about 90 minutes, at least about 120 minutes, at least about 150 minutes, at least about 240 minutes, from about 0.01 seconds to about 240 minutes, from about 0.5 seconds to about 240 minutes, from about 1 second to about 240 minutes, from about 1 minute to about 240 minutes, from about 0.01 seconds to about 60 minutes, from about 0.5 seconds to about 60 minutes, from about 1 second to about 60 minutes, from about 1 minute to about 60 minutes, from about 0.01 seconds to about 10 minutes, from about 0.5 seconds to about 10 minutes, from about 1 second to about 10 minutes, from about 1 minute to about 10 minutes, from about 0.01 seconds to about 1 minute, from about 0.5 seconds to about 1 minute, from about 1 second to about 1 minute, no more than about 600 minutes, no more than about 450 minutes, no more than about 300 minutes, no more than about 180 minutes, no more than about 120 minutes, no more than about 90 minutes, no more than about 60 minutes, no more than about 30 minutes, no more than about 15 minutes, no more than about 10 minutes, no more than about 5 minutes, no more than about 1 minute, no more than about 30 seconds, no more than about 10 seconds, or no more than about 1 second. During the course of heating, the temperature can vary.

Examples of the rate of heating include at least about 120° C./min, at least about 200° C./min, at least about 300° C./min, at least about 400° C./min, at least about 600° C./min, at least about 800° C./min, at least about 1000° C./min, at least about 1200° C./min, at least about 1500° C./min, at least about 1800° C./min, and at least about 2000° C./min.

Graphene sheets can be annealed or reduced to graphene sheets having higher carbon to oxygen ratios by heating under reducing atmospheric conditions (e.g., in systems purged with inert gases or hydrogen). Reduction/annealing temperatures are preferably at least about 300° C., or at least about 350° C., or at least about 400° C., or at least about 500° C., or at least about 600° C., or at least about 750° C., or at least about 850° C., or at least about 950° C., or at least about 1000° C. The temperature used can be, for example, between about 750 about and 3000° C., or between about 850 and 2500° C., or between about 950 and about 2500° C.

The time of heating can be for example, at least about 1 second, or at least about 10 second, or at least about 1 minute, or at least about 2 minutes, or at least about 5 minutes. In some embodiments, the heating time will be at least about 15 minutes, or about 30 minutes, or about 45 minutes, or about 60 minutes, or about 90 minutes, or about 120 minutes, or about 150 minutes. During the course of annealing/reduction, the temperature can vary within these ranges.

The heating can be done under a variety of conditions, including in an inert atmosphere (such as argon or nitrogen) or a reducing atmosphere, such as hydrogen (including hydrogen diluted in an inert gas such as argon or nitrogen), or under vacuum. The heating can be done in any appropriate vessel, such as a fused silica or a mineral or ceramic vessel or a metal vessel. The materials being heated including any starting materials and any products or intermediates) can be contained in an essentially constant location in single batch reaction vessel, or can be transported through one or more vessels during the reaction in a continuous or batch reaction. Heating can be done using any suitable means, including the use of furnaces and infrared heaters.

The graphene sheets preferably have a surface area of at least about 100 m²/g to, or of at least about 200 m²/g, or of at least about 300 m²/g, or of least about 350 m²/g, or of least about 400 m²/g, or of least about 500 m²/g, or of least about 600 m²/g., or of least about 700 m²/g, or of least about 800 m²/g, or of least about 900 m²/g, or of least about 700 m²/g. The surface area can be about 400 to about 1100 m²/g. The theoretical maximum surface area can be calculated to be 2630 m²/g. The surface area includes all values and subvalues therebetween, especially including 400, 500, 600, 700, 800, 900, 1000, 1100, 1200, 1300, 1400, 1500, 1600, 1700, 1800, 1900, 2000, 2100, 2200, 2300, 2400, 2500, and 2630 m²/g.

The graphene sheets can have number average aspect ratios of about 100 to about 100,000, or of about 100 to about 50,000, or of about 100 to about 25,000, or of about 100 to about 10,000 (where “aspect ratio” is defined as the ratio of the longest dimension of the sheet to the shortest).

Surface area can be measured using either the nitrogen adsorption/BET method at 77 K or a methylene blue (MB) dye method in liquid solution.

The dye method is carried out as follows: A known amount of graphene sheets is added to a flask. At least 1.5 g of MB are then added to the flask per gram of graphene sheets. Ethanol is added to the flask and the mixture is ultrasonicated for about fifteen minutes. The ethanol is then evaporated and a known quantity of water is added to the flask to re-dissolve the free MB. The undissolved material is allowed to settle, preferably by centrifuging the sample. The concentration of MB in solution is determined using a UV-vis spectrophotometer by measuring the absorption at λₘₐₓ=298 nm relative to that of standard concentrations.

The difference between the amount of MB that was initially added and the amount present in solution as determined by UV-vis spectrophotometry is assumed to be the amount of MB that has been adsorbed onto the surface of the graphene sheets. The surface area of the graphene sheets are then calculated using a value of 2.54 m² of surface covered per one mg of MB adsorbed.

The graphene sheets can have a bulk density of from about 0.01 to at least about 200 kg/m³. The bulk density includes all values and subvalues therebetween, especially including 0.05, 0.1, 0.5, 1, 5, 10, 15, 20, 25, 30, 35, 50, 75, 100, 125, 150, and 175 kg/m³.

Graphene sheets can be used in a dry or powder form (with little or no solvent), as a blend/dispersion/etc. in one or more solvents.

The graphene sheets can be functionalized with, for example, oxygen-containing functional groups (including, for example, hydroxyl, carboxyl, and epoxy groups) and typically have an overall carbon to oxygen molar ratio (C/O ratio), as determined by bulk elemental analysis, of at least about 1:1, or more preferably, at least about 3:2. Examples of carbon to oxygen ratios include about 3:2 to about 85:15; about 3:2 to about 20:1; about 3:2 to about 30:1; about 3:2 to about 40:1; about 3:2 to about 60:1; about 3:2 to about 80:1; about 3:2 to about 100:1; about 3:2 to about 200:1; about 3:2 to about 500:1; about 3:2 to about 1000:1; about 3:2 to greater than 1000:1; about 10:1 to about 30:1; about 80:1 to about 100:1; about 20:1 to about 100:1; about 20:1 to about 500:1; about 20:1 to about 1000:1; about 50:1 to about 300:1; about 50:1 to about 500:1; and about 50:1 to about 1000:1. In some embodiments, the carbon to oxygen ratio is at least about 10:1, or at least about 15:1, or at least about 20:1, or at least about 35:1, or at least about 50:1, or at least about 75:1, or at least about 100:1, or at least about 200:1, or at least about 300:1, or at least about 400:1, or at least 500:1, or at least about 750:1, or at least about 1000:1; or at least about 1500:1, or at least about 2000:1. The carbon to oxygen ratio also includes all values and subvalues between these ranges.

The graphene sheets can contain atomic scale kinks. These kinks can be caused by the presence of lattice defects in, or by chemical functionalization of the two-dimensional hexagonal lattice structure of the graphite basal plane.

The compositions can further comprise graphite (including natural, Kish, and synthetic, annealed, pyrolytic, highly oriented pyrolytic, etc. graphites). In some cases, the graphite can be present in from about 1 to about 99 percent, or from about 10 to about 99 percent, or from about 20 to about 99 percent, from about 30 to about 99 percent, or from about 40 to about 99 percent, or from about 50 to about 99 percent, or from about 60 to about 99 percent, or from about 70 to about 99 percent, or from about 80 to about 99 percent, or from about 85 to about 99 percent, or from about 90 to about 99 percent, or from about 1 to about 95 percent, or from about 10 to about 95 percent, or from about 20 to about 95 percent, from about 30 to about 95 percent, or from about 40 to about 95 percent, or from about 50 to about 95 percent, or from about 60 to about 95 percent, or from about 70 to about 95 percent, or from about 80 to about 95 percent, or from about 85 to about 95 percent, or from about 90 to about 95 percent, or from about 1 to about 80 percent, or from about 10 to about 80 percent, or from about 20 to about 80 percent, from about 30 to about 80 percent, or from about 40 to about 80 percent, or from about 50 to about 80 percent, or from about 60 to about 80 percent, or from about 70 to about 80 percent, or from about 1 to about 70 percent, or from about 10 to about 70 percent, or from about 20 to about 70 percent, from about 30 to about 70 percent, or from about 40 to about 70 percent, or from about 50 to about 70 percent, or from about 60 to about 70 percent, or from about 1 to about 60 percent, or from about 10 to about 60 percent, or from about 20 to about 60 percent, from about 30 to about 60 percent, or from about 40 to about 60 percent, or from about 50 to about 60 percent, or from about 1 to about 50 percent, or from about 10 to about 50 percent, or from about 20 to about 50 percent, from about 30 to about 50 percent, or from about 40 to about 50 percent, or from about 1 to about 40 percent, or from about 10 to about 40 percent, or from about 20 to about 40 percent, from about 30 to about 40 percent, from about 1 to about 30 percent, or from about 10 to about 30 percent, or from about 20 to about 30 percent, or from about 1 to about 20 percent, or from about 10 to about 20 percent, or from about 1 to about 10 percent, based on the total weight of graphene sheets and graphite.

The graphene sheets can comprise two or more graphene powders having different particle size distributions and/or morphologies. The graphite can also comprise two or more graphite powders having different particle size distributions and/or morphologies.

If one or more additional thermally and/or electrically conductive additives are used, they can be present in the composition in from about 1 to about 99 weight percent, or about 5 to about 95 weight percent, or about 5 to about 80 weight percent, or about 5 to about 70 weight percent, or about 5 to about 50 weight percent, or about 5 to about 35 weight percent, or about 15 to about 99 weight percent, or about 15 to about 95 weight percent, or about 15 to about 80 weight percent, or about 15 to about 70 weight percent, or about 15 to about 50 weight percent, or about 15 to about 35 weight percent, or about 30 to about 99 weight percent, or about 30 to about 95 weight percent, or about 30 to about 80 weight percent, or about 30 to about 70 weight percent, or about 30 to about 50 weight percent, or about 50 to about 99 weight percent, or about 50 to about 95 weight percent, or about 50 to about 80 weight percent, or about 50 to about 70 weight percent, or about 70 to about 99 weight percent, or about 70 to about 95 weight percent, or about 70 to about 80 weight percent, or about 80 to about 99 weight percent, or about 80 to about 95 weight percent, or about 90 to about 99 weight percent, or about 90 to about 95 weight percent, based on the total weight of the conductive additive and graphene sheets or graphene sheet and graphite, if present.

They graphene sheets and other components can be combined with polymers to make composites, inks and coatings, and the like. They can be dispersed in one or more solvents with or without a polymer binder.

The compositions can be in the form of polymer composites, such as those made from thermoplastics and thermosetting polymers. The compositions can be in the form of inks and coatings. By the terms “ink” and “coating” are meant composition that are in a form that is suitable for application to a substrate as well as the material after it is applied to the substrate, while it is being applied to the substrate, and both before and after any post-application treatments (such as evaporation, cross-linking, curing, etc.). The components of the ink and coating compositions can vary during these stages. The inks and coatings can optionally further comprise at least one polymeric binder. They can be in the form of paints.

When used, the polymer binders can be thermosets, thermoplastics, non-melt processible polymers, etc. Polymers can also comprise monomers that can be polymerized before, during, or after the application of the coating to the substrate. Polymeric binders can be crosslinked or otherwise cured after the coating has been applied to the substrate. Examples of polymers include, but are not limited to acrylic polymers, polyolefins (such as polyethylene, linear low density polyethylene (LLDPE), low density polyethylene (LDPE), high density polyethylene, polypropylene, and olefin copolymers), styrene/butadiene rubbers (SBR), styrene/ethylene/butadiene/styrene copolymers (SEBS), butyl rubbers, ethylene/propylene copolymers (EPR), ethylene/propylene/diene monomer copolymers (EPDM), polystyrene (including high impact polystyrene), poly(vinyl acetates), ethylene/vinyl acetate copolymers (EVA), poly(vinyl alcohols), ethylene/vinyl alcohol copolymers (EVOH), poly(vinyl butyral) (PVB), poly(vinyl formal), poly(methyl methacrylate) and other acrylate polymers and copolymers (such as methyl methacrylate polymers, methacrylate copolymers, polymers derived from one or more acrylates, methacrylates, ethyl acrylates, ethyl methacrylates, butyl acrylates, butyl methacrylates, glycidyl acrylates and methacrylates and the like), olefin and styrene copolymers, acrylonitrile/butadiene/styrene (ABS), styrene/acrylonitrile polymers (SAN), styrene/maleic anhydride copolymers, isobutylene/maleic anhydride copolymers, ethylene/acrylic acid copolymers, poly(acrylonitrile), poly(vinyl acetate) and poly(vinyl acetate) copolymers, poly(vinyl pyrrolidone) and poly(vinyl pyrrolidone) copolymers, vinyl acetate and vinyl pyrrolidone copolymers, polycarbonates (PC), polyamides, polyesters, liquid crystalline polymers (LCPs), poly(lactic acid) (PLA), poly(phenylene oxide) (PPO), PPO-polyamide alloys, polysulphones (PSU), polysulfides, polyetherketone (PEK), polyetheretherketone (PEEK), polyimides, polyoxymethylene (POM) homo- and copolymers, polyetherimides, fluorinated ethylene propylene polymers (FEP), poly(vinyl fluoride), poly(vinylidene fluoride), poly(vinylidene chloride), and poly(vinyl chloride), polyurethanes (thermoplastic and thermosetting (including crosslinked polyurethanes such as those crosslinked with amines, etc.), aramides (such as Kevlar® and Nomex®), polysulfides, polytetrafluoroethylene (PTFE), polysiloxanes (including polydimethylenesiloxane, dimethylsiloxane/vinylmethylsiloxane copolymers, vinyldimethylsiloxane terminated poly(dimethylsiloxane), etc.), elastomers, epoxy polymers (including crosslinked epoxy polymers such as those crosslinked with polysulfones, amines, etc.), decalin polymers, polyureas, alkyds, cellulosic polymers (such as nitrocellulose, ethyl cellulose, ethyl hydroxyethyl cellulose, carboxymethyl cellulose, cellulose acetate, cellulose acetate propionates, and cellulose acetate butyrates), polyethers (such as poly(ethylene oxide), poly(propylene oxide), poly(propylene glycol), oxide/propylene oxide copolymers, etc.), acrylic latex polymers, polyester acrylate oligomers and polymers, polyester diol diacrylate polymers, UV-curable resins, etc.

Examples of elastomers include, but are not limited to, polyurethanes, copolyetheresters, rubbers (including butyl rubbers and natural rubbers), styrene/butadiene copolymers, styrene/ethylene/butadiene/styrene copolymer (SEBS), polyisoprene, ethylene/propylene copolymers (EPR), ethylene/propylene/diene monomer copolymers (EPDM), polysiloxanes, and polyethers (such as poly(ethylene oxide), poly(propylene oxide), and their copolymers).

Examples of polyamides include, but are not limited to, aliphatic polyamides (such as polyamide 4,6; polyamide 6,6; polyamide 6; polyamide 11; polyamide 12; polyamide 6,9; polyamide 6,10; polyamide 6,12; polyamide 10,10; polyamide 10,12; and polyamide 12,12), alicyclic polyamides, and aromatic polyamides (such as poly(m-xylylene adipamide) (polyamide MXD,6)) and polyterephthalamides such as poly(dodecamethylene terephthalamide) (polyamide 12,T), poly(decamethylene terephthalamide) (polyamide 10,T), poly(nonamethylene terephthalamide) (polyamide 9,T), the polyamide of hexamethylene terephthalamide and hexamethylene adipamide, the polyamide of hexamethyleneterephthalamide, and 2-methylpentamethyleneterephthalamide), etc. The polyamides can be polymers and copolymers (i.e., polyamides having at least two different repeat units) having melting points between about 120 and 255° C. including aliphatic copolyamides having a melting point of about 230° C. or less, aliphatic copolyamides having a melting point of about 210° C. or less, aliphatic copolyamides having a melting point of about 200° C. or less, aliphatic copolyamides having a melting point of about 180° C. or less, etc. Examples of these include those sold under the trade names Macromelt by Henkel and Versamid by Cognis.

Examples of acrylate polymers include those made by the polymerization of one or more acrylic acids (including acrylic acid, methacrylic acid, etc.) and their derivatives, such as esters. Examples include methyl acrylate polymers, methyl methacrylate polymers, and methacrylate copolymers. Examples include polymers derived from one or more acrylates, methacrylates, acrylic acid, methacrylic acid, methyl acrylate, methyl methacrylate, ethyl acrylate, ethyl methacrylate, butyl acrylate, butyl methacrylate, glycidyl acrylate, glycidyl methacrylates, 2-ethylhexyl acrylate, 2-ethylhexyl methacrylate, hydroxyethyl acrylate, hydroxyethyl (meth)acrylate, acrylonitrile, and the like. The polymers can comprise repeat units derived from other monomers such as olefins (e.g. ethylene, propylene, etc.), vinyl acetates, vinyl alcohols, vinyl pyrrolidones, etc. They can include partially neutralized acrylate polymers and copolymers (such as ionomer resins).

Examples of polymers include Elvacite® polymers supplied by Lucite International, Inc., including Elvacite® 2009, 2010, 2013, 2014, 2016, 2028, 2042, 2045, 2046, 2550, 2552, 2614, 2669, 2697, 2776, 2823, 2895, 2927, 3001, 3003, 3004, 4018, 4021, 4026, 4028, 4044, 4059, 4400, 4075, 4060, 4102, etc. Other polymer families include Bynel® polymers (such as Bynel® 2022 supplied by DuPont) and Joncryl® polymers (such as Joncryl® 678 and 682).

Examples of polyesters include, but are not limited to, poly(butylene terephthalate) (PBT), poly(ethylene terephthalate) (PET), poly(1,3-propylene terephthalate) (PPT), poly(ethylene naphthalate) (PEN), poly(cyclohexanedimethanol terephthalate) (PCT)), etc.

In some embodiment, the polymer has a acid number of at least about 5, or at least about 10, or at least about 15, or at least about 20.

In some embodiments, the glass transition temperature of at least one polymer is no greater than about 100° C., 90° C., or no greater than about 80° C., or no greater than about 70° C., or no greater than about 60° C., or no greater than about 50° C., or no greater than about 40° C.

Examples of solvents into which the graphene sheets and, optionally, other components can be dispersed include water, distilled or synthetic isoparaffinic hydrocarbons (such Isopar® and Norpar® (both manufactured by Exxon) and Dowanol® (manufactured by Dow), citrus terpenes and mixtures containing citrus terpenes (such as Purogen, Electron, and Positron (all manufactured by Ecolink)), terpenes and terpene alcohols (including terpineols, including alpha-terpineol), limonene, aliphatic petroleum distillates, alcohols (such as methanol, ethanol, n-propanol, i-propanol, n-butanol, butanol, sec-butanol, tert-butanol, pentanols, i-amyl alcohol, hexanols, heptanols, octanols, diacetone alcohol, butyl glycol, etc.), ketones (such as acetone, methyl ethyl ketone, cyclohexanone, i-butyl ketone, 2,6,8,trimethyl-4-nonanone etc.), esters (such as methyl acetate, ethyl acetate, n-propyl acetate, i-propyl acetate, n-butyl acetate, i-butyl acetate, tert-butyl acetate, carbitol acetate, etc.), glycol ethers, ester and alcohols (such as 2-(2-ethoxyethoxy)ethanol, propylene glycol monomethyl ether and other propylene glycol ethers; ethylene glycol monobutyl ether, 2-methoxyethyl ether (diglyme), propylene glycol methyl ether (PGME); and other ethylene glycol ethers; ethylene and propylene glycol ether acetates, diethylene glycol monoethyl ether acetate, 1-methoxy-2-propanol acetate (PGMEA); and hexylene glycol (such as Hexasol™ (supplied by SpecialChem)), dibasic esters (such as dimethyl succinate, dimethyl glutarate, dimethyl adipate), dimethylsulfoxide (DMSO), 1,3-dimethyl-3,4,5,6-tetrahydro-2(1H)-pyrimidinone (DMPU), imides, amides (such as dimethylformamide (DMF), dimethylacetamide, etc.), cyclic amides (such as N-methylpyrrolidone and 2-pyrrolidone), lactones (such as beta-propiolactone, gamma-valerolactone, delta-valerolactone, gamma-butyrolactone, epsilon-caprolactone), cyclic imides (such as imidazolidinones such as N,N′-dimethylimidazolidinone (1,3-dimethyl-2-imidazolidinone)) (DMI), aromatic solvents and aromatic solvent mixtures (such as toluene, xylenes, mesitylene, cumene, etc.), petroleum distillates, naphthas (such as VM&P naphtha),and mixtures of two or more of the foregoing and mixtures of one or more of the foregoing with other carriers. Solvents can be low- or non-VOC solvents, non-hazardous air pollution solvents, and non-halogenated solvents.

The compositions can contain additives such as dispersion aids (including surfactants, emulsifiers, and wetting aids), adhesion promoters, thickening agents (including clays), defoamers and antifoamers, biocides, additional fillers, flow enhancers, stabilizers, crosslinking and curing agents, conductive additives, etc.

Examples of dispersing aids include glycol ethers (such as poly(ethylene oxide), block copolymers derived from ethylene oxide and propylene oxide (such as those sold under the trade name Pluronic® by BASF), acetylenic diols (such as 2,5,8,11-tetramethyl-6-dodecyn-5,8-diol ethoxylate and others sold by Air Products under the trade names Surfynol® and Dynol®), salts of carboxylic acids (including alkali metal and ammonium salts), and polysiloxanes.

Examples of grinding aids include stearates (such as Al, Ca, Mg, and Zn stearates) and acetylenic diols (such as those sold by Air Products under the trade names Surfynol® and Dynol®).

Examples of adhesion promoters include titanium chelates and other titanium compounds such as titanium phosphate complexes (including butyl titanium phosphate), titanate esters, diisopropoxy titanium bis(ethyl-3-oxobutanoate, isopropoxy titanium acetylacetonate, and others sold by Johnson-Matthey Catalysts under the trade name Vertec.

The compositions can optionally comprise at least one “multi-chain lipid”, by which term is meant a naturally-occurring or synthetic lipid having a polar head group and at least two nonpolar tail groups connected thereto. Examples of polar head groups include oxygen-, sulfur-, and halogen-containing, phosphates, amides, ammonium groups, amino acids (including α-amino acids), saccharides, polysaccharides, esters (Including glyceryl esters), zwitterionic groups, etc.

The tail groups can be the same or different. Examples of tail groups include alkanes, alkenes, alkynes, aromatic compounds, etc. They can be hydrocarbons, functionalized hydrocarbons, etc. The tail groups can be saturated or unsaturated. They can be linear or branched. The tail groups can be derived from fatty acids, such as oleic acid, palmitic acid, stearic acid, arachidic acid, erucic acid, arachadonic acid, linoleic acid, linolenic acid, oleic acid, etc.

Examples of multi-chain lipids include, but are not limited to, lecithin and other phospholipids (such as phosphatidylcholine, phosphoglycerides (including phosphatidylserine, phosphatidylinositol, phosphatidylethanolamine (cephalin), and phosphatidylglycerol) and sphingomyelin); glycolipids (such as glucosyl-cerebroside); saccharolipids; sphingolipids (such as ceramides, di- and triglycerides, phosphosphingolipids, and glycosphingolipids); etc. They can be amphoteric, including zwitterionic.

Examples of thickening agents include glycol ethers (such as poly(ethylene oxide), block copolymers derived from ethylene oxide and propylene oxide (such as those sold under the trade name Pluronic® by BASF), long-chain carboxylate salts (such aluminum, calcium, zinc, etc. salts of stearates, oleats, palmitates, etc.), aluminosilicates (such as those sold under the Minex® name by Unimin Specialty Minerals and Aerosil® 9200 by Evonik Degussa), fumed silica, natural and synthetic zeolites, etc.

Examples of crosslinking agents include radical initiators such as radical polymerization initiators, radical sources, etc., including organic and inorganic compounds. Coagents and crosslinking promoters may be used as well. Examples include organic and inorganic peroxides (such as hydrogen peroxide, dialkyl peroxides, hydroperoxides, peracids, diacyl peroxides, peroxy esters, ketone peroxides, hydrocarbon peroxides, organometallic peroxides, organic polyoxides, organic polyoxides, dialkyl trioxides, hydrotrioxides, tetroxides, alkali metal peroxides (such as lithium peroxide), etc.), azo compounds, polyphenylhydrocarbons, substituted hydrazines, alkoxyamines, nitrocompounds, nitrates, nitrites, nitroxides, disulfides, polysulfides, persulfates (e.g. potassium persulfate, etc.), etc.

Examples of peroxides include, but are not limited to dibenzoyl peroxide, dicumyl peroxide, acetone peroxide, methyl ethyl ketone peroxide, lauroyl peroxide, tert-butyl peroxide, tert-butyl peracetate, di-tert-amyl peroxide, tert-butyl hydroperoxide, cumene hydroperoxide, 1,3-bis-(tert-butylperoxy-1-propyl) benzene, bis-(tert-butylperoxy) valerate, bis-(2,4-dichlorobenzoyl) peroxide, etc.

Examples of azo compounds include azobisisobutylonitrile (AIBN); 1,1′-azobis(cyclohexanecarbonitrile) (ABCN); 2,2′-azobis(2-methylbutyronitrile); 2,2′-azobis(2-methyl propionitrile); 2,2′-azobis(2-methyl propionitrile); N-tert-butyl-N-(2-methyl-1-phenylpropyl)-O-(1-phenylethyl)hydroxylamine, etc.

In one embodiment, graphene sheets are used without a binder or with minimal amounts of a binder when used with a crosslinking agent.

The compositions can be made using any suitable method, including wet or dry methods and batch, semi-continuous, and continuous methods. They can be made using melt-processing methods (using, for example, a single or twin-screw extruder, a blender, a kneader, a Banbury mixer, etc.) and solution/dispersion blending. Dispersions, suspensions, solutions, etc. of graphene sheets and thermally conductive additives (including inks and coatings formulations) can be made or mechanically processed (e.g., milled/ground, blended, dispersed, suspended, etc.) by using suitable mixing, dispersing, stirring, and/or compounding techniques.

Components of the compositions, such as one or more of the graphene sheets, conductive additives (if used), graphite (if used), binders, carriers, and/or other components can be processed (e.g., milled/ground, blended, etc. by using suitable mixing, dispersing, and/or compounding techniques and apparatus, including ultrasonic devices, high-shear mixers, ball mills, attrition equipment, sandmills, two-roll mills, three-roll mills, cryogenic grinding crushers, extruders, kneaders, double planetary mixers, triple planetary mixers, high pressure homogenizers, horizontal and vertical wet grinding mills, etc.) Processing (including grinding) technologies can be wet or dry and can be continuous or discontinuous. Suitable materials for use as grinding media include metals, carbon steel, stainless steel, ceramics, stabilized ceramic media (such as cerium yttrium stabilized zirconium oxide), PTFE, glass, tungsten carbide, etc. Methods such as these can be used to change the particle size and/or morphology of the graphite, graphene sheets, thermally conductive additives, other components, and blends of two or more components.

Components can be processed together or separately and can go through multiple processing (including mixing/blending) stages, each involving one or more components (including blends).

There is no particular limitation to the way in which the graphene sheets, graphite (if used), additives (if used), and other components are processed and combined. For example, graphene sheets and/or graphite can be processed into given particle size distributions and/or morphologies separately and then combined for further processing with or without the presence of additional components. Unprocessed graphene sheets and/or graphite can be combined with processed graphene sheets and/or graphite and further processed with or without the presence of additional components. Processed and/or unprocessed graphene sheets and/or processed and/or unprocessed graphite can be combined with other components, such as one or more binders and then combined with processed and/or unprocessed graphene sheets and/or processed and/or unprocessed graphite. Two or more combinations of processed and/or unprocessed graphene sheets and/or processed and/or unprocessed graphite that have been combined with other components can be further combined or processed.

Graphene sheets and/or graphite can be processed (e.g. milled or ground) in the presence of the metal particles, or the graphene and/or graphite (if present) can be processed separately from some or all of the thermally conductive additives, and the components later blended. The graphene sheets and/or graphite and/or thermally conductive additives can be separately processed in the presence of binders and then later combined.

In one embodiment, if a multi-chain lipid is used, it can be added to graphene sheets (and/or graphite if present) before processing.

After blending and/or grinding steps, additional components can be added to the compositions, including, but not limited to, thickeners, viscosity modifiers, binders, etc. The compositions can also be diluted by the addition of more carrier.

Inks and coatings can be formed by blending the graphene sheets with at least one solvent and/or binder, and, optionally, other additives. Blending can be done using one or more of the preceding methods.

The compositions can be formed by polymerizing monomers in the presense of graphene sheets and, optionally, other additives.

Polymer composite compositions can be made using any suitable melt-mixing method, such as using a single or twin-screw extruder, a blender, a kneader, or a Banbury mixer. In one embodiment of the invention, the compositions are melt-mixed blends wherein the non-polymeric ingredients are well-dispersed in the polymer matrix, such that the blend forms a unified whole.

Polymer composite compositions may be formed into thermal management devices using any suitable technique, including compression molding, extrusion, ram extrusion, injection molding, extrusion, co-extrusion, rotational molding, blow molding, injection blow molding, thermoforming, vacuum forming, casting, solution casting, centrifugal casting, overmolding, resin transfer molding, vacuum assisted resin transfer molding, spinning, printing, etc. Thermoset compositions can be formed by mixing resin precursors with graphene sheets and, optionally, other additives in a mold and curing.

Inks and coatings can be applied to a wide variety of substrates to form the thermal management device, including, but not limited to, flexible and/or stretchable materials, silicones and other elastomers and other polymeric materials, metals (such as aluminum, copper, steel, stainless steel, etc.), adhesives, heat-sealable materials (such as cellulose, biaxially oriented polypropylene (BOPP), poly(lactic acid), polyurethanes, etc.), fabrics (including cloths) and textiles (such as cotton, wool, polyesters, rayon, etc.), clothing, glasses and other minerals, ceramics, silicon surfaces, wood, paper, cardboard, paperboard, cellulose-based materials, glassine, labels, silicon and other semiconductors, laminates, corrugated materials, concrete, bricks, fiber-reinforced materials (such as glass fiber reinforced materials, glass fiber-reinforced epoxy resins, fiberglass, etc.), fiber mats, paper-reinforced phenolic resins, building materials, etc. Substrates can in the form of films, papers, wafers, larger three-dimensional objects, etc.

The substrates can have been treated with other coatings (such as paints) or similar materials before the inks and coatings are applied. Examples include substrates (such as PET) coated with indium tin oxide, antimony tin oxide, etc. They can be woven, nonwoven, in mesh form; etc. They can be woven, nonwoven, in mesh form; etc.

The substrates can be paper-based materials generally (including paper, paperboard, cardboard, glassine, etc.). Paper-based materials can be surface treated, impregnated, etc. Examples of surface treatments include coatings such as polymeric coatings, which can include PET, polyethylene, polypropylene, biaxially oriented polypropylene (BOPP), acetates, nitrocellulose, etc. Coatings can be adhesives. Paper based materials can be sized.

Examples of polymeric materials include, but are not limited to, those comprising thermoplastics and thermosets, including elastomers and rubbers (including thermoplastics and thermosets, phenolic resins, paper-reinforced phenolic resins, silicones, fluorinated polysiloxanes, natural rubber, butyl rubber, chlorosulfonated polyethylene, chlorinated polyethylene, styrene/butadiene copolymers (SBR), styrene/ethylene/butadiene/stryene copolymers (SEBS), styrene/ethylene/butadiene/stryene copolymers grafted with maleic anhydride, styrene/isoprene/styrene copolymers (SIS), polyisoprene, nitrile rubbers, hydrogenated nitrile rubbers, neoprene, ethylene/propylene copolymers (EPR), ethylene/propylene/diene copolymers (EPDM), ethylene/vinyl acetate copolymer (EVA), hexafluoropropylene/vinylidene fluoride/tetrafluoroethylene copolymers, tetrafluoroethylene/propylene copolymers, fluorelastomers, polyesters (such as poly(ethylene terephthalate), poly(butylene terephthalate), poly(ethylene naphthalate), liquid crystalline polyesters, poly(lactic acid), etc).; polystyrene; polyamides (including polyterephthalamides); polyimides (such as Kapton®); aramids (such as Kevlar® and Nomex®); fluoropolymers (such as fluorinated ethylene propylene (FEP), polytetrafluoroethylene (PTFE), poly(vinyl fluoride), poly(vinylidene fluoride), etc.); polyetherimides; poly(vinyl chloride); poly(vinylidene chloride); polyurethanes (such as thermoplastic polyurethanes (TPU); spandex, cellulosic polymers (such as cellulose, nitrocellulose, cellulose acetate, etc.); styrene/acrylonitriles polymers (SAN); arcrylonitrile/butadiene/styrene polymers (ABS); polycarbonates; polyacrylates; poly(methyl methacrylate); ethylene/vinyl acetate copolymers; thermoset epoxies and polyurethanes; polyolefins (such as polyethylene (including low density polyethylene, high density polyethylene, ultrahigh molecular weight polyethylene, etc.), polypropylene (such as biaxially-oriented polypropylene, etc.); Mylar; etc. They can be non-woven materials, such as DuPont Tyvek®. They can be adhesive or adhesive-backed materials (such as adhesive-backed papers or paper substitutes). They can be mineral-based paper substitutes such as Teslin® from PPG Industries. The substrate can be a transparent or translucent or optical material, such as glass, quartz, polymer (such as polycarbonate or poly(meth)acrylates (such as poly(methyl methacrylate).

The inks and coatings can be applied to the substrate using any suitable method, including, but not limited to, painting, pouring, spin casting, solution casting, dip coating, powder coating, by syringe or pipette, spray coating, curtain coating, lamination, co-extrusion, electrospray deposition, ink-jet printing, spin coating, thermal transfer (including laser transfer) methods, doctor blade printing, screen printing, rotary screen printing, gravure printing, lithographic printing, intaglio printing, digital printing, capillary printing, offset printing, electrohydrodynamic (EHD) printing (a method of which is described in WO 2007/053621, which is hereby incorporated herein by reference), microprinting, pad printing, tampon printing, stencil printing, wire rod coating, drawing, flexographic printing, stamping, xerography, microcontact printing, dip pen nanolithography, laser printing, via pen, via brush, via sponge, or similar means, etc. The compositions can be applied in multiple layers.

After they have been applied to a substrate, the inks and coatings can be cured using any suitable technique, including drying and oven-drying (in air or another inert or reactive atmosphere), UV curing, IR curing, drying, crosslinking, thermal curing, laser curing, IR curing, microwave curing or drying, sintering, and the like.

The cured inks and coatings can have a variety of thicknesses. For example, they can optionally have a thickness of at least about 2 nm, or at least about 5 nm. In various embodiments, the coatings can optionally have a thickness of about 2 nm to 2 mm, about 5 nm to 1 mm, about 2 nm to about 100 nm, about 2 nm to about 200 nm, about 2 nm to about 500 nm, about 2 nm to about 1 micrometer, about 5 nm to about 200 nm, about 5 nm to about 500 nm, about 5 nm to about 1 micrometer, about 5 nm to about 50 micrometers, about 5 nm to about 200 micrometers, about 10 nm to about 200 nm, about 50 nm to about 500 nm, about 50 nm to about 1 micrometer, about 100 nm to about 10 micrometers, about 1 micrometer to about 2 mm, about 1 micrometer to about 1 mm, about 1 micrometer to about 500 micrometers, about 1 micrometer to about 200 micrometers, about 1 micrometer to about 100 micrometers, about 50 micrometers to about 1 mm, about 100 micrometers to about 2 mm, about 100 micrometers to about 1 mm, about 100 micrometers to about 750 micrometers, about 100 micrometers to about 500 micrometers, about 500 micrometers to about 2 mm, or about 500 micrometers to about 1 mm.

When applied to a substrate, the inks and coatings can have a variety of forms. They can be present as a film or lines, patterns, letters, numbers, circuitry, logos, identification tags, and other shapes and forms. The inks and coatings can be covered in whole or in part with additional material, such as overcoatings, varnishes, polymers, fabrics, etc.

The inks and coatings can be applied to the same substrate in varying thicknesses at different points and can be used to build up three-dimensional structures on the substrate.

The compositions, including those in the form of polymer composites, dispersions, inks and coatings, etc. can be electrically and/or thermally conductive. In some embodiments, the composition can have a conductivity of at least about 10⁻⁸ S/m. It can have a conductivity of about 10⁻⁶ S/m to about 10⁵ S/m, or of about 10⁻⁵ S/m to about 10⁵ S/m. In other embodiments of the invention, the coating has conductivities of at least about 0.001 S/m, of at least about 0.01 S/m, of at least about 0.1 S/m, of at least about 1 S/m, of at least about 10 S/m, of at least about 100 S/m, or at least about 1000 S/m, or at least about 10,000 S/m, or at least about 20,000 S/m, or at least about 30,000 S/m, or at least about 40,000 S/m, or at least about 50,000 S/m, or at least about 60,000 S/m, or at least about 75,000 S/m, or at least about 10⁵ S/m, or at least about 10⁶ S/m.

In some embodiments, the surface resistivity of the composition (including polymer composites, cured inks and coatings, etc.) can be no greater than about 10 megaΩ/square/mil, or no greater than about 1 mega Ω/square/mil, or no greater than about 500 kiloΩ/square/mil, or no greater than about 200 kiloΩ/square/mil, or no greater than about 100 kiloΩ/square/mil, or no greater than about 50 kiloΩ/square/mil, or no greater than about 25 kiloΩ/square/mil, or no greater than about 10 kiloΩ/square/mil, or no greater than about 5 kilo Ω/square/mil, or no greater than about 1000 Ω/square/mil, or no greater than about 700 Ω/square/mil, or no greater than about 500 Ω/square/mil, or no greater than about 350 Ω/square/mil, or no greater than about 200 Ω/square/mil, or no greater than about 200 Ω/square/mil, or no greater than about 150 Ω/square/mil, or no greater than about 100 Ω/square/mil, or no greater than about 75 Ω/square/mil, or no greater than about 50 Ω/square/mil, or no greater than about 30 Ω/square/mil, or no greater than about 20 Ω/square/mil, or no greater than about 10 Ω/square/mil, or no greater than about 5 Ω/square/mil, or no greater than about 1 Ω/square/mil, or no greater than about 0.1 Ω/square/mil, or no greater than about 0.01 Ω/square/mil, or no greater than about 0.001 Ω/square/mil.

In some embodiments, the composition can have a thermal conductivity of about 0.1 to about 50 W/m·K, or of about 0.5 to about 30 W/m·K, or of about 0.1 to about 0.5 W/m·K, or of about 0.1 to about 1 W/m·K, or of about 0.1 to about 5 W/m·K, or of about 0.5 to about 2 W/m·K, or of about 1 to about 5 W/m·K, or of about 0.1 to about 0.5 W/m·K, or of about 0.1 to about 50 W/m·K, or of about 1 to about 30 W/m·K, or of about 1 to about 20 W/m·K, or of about 1 to about 10 W/m·K, or of about 1 to about 5 W/m·K, or of about 2 to about 25 W/m·K, or of about 5 to about 25 W/m·K, or of at least about 0.7 W/m·K, or of at least 1 W/m·K, or of at least 1.5 W/m·K, or of at least 3 W/m·K, or of at least 5 W/m·K, or of at least 7 W/m·K, or of at least 10 W/m·K, or of at least 15 W/m·K.

1. A thermal management device system comprising a thermal management device component and a computing device component, wherein the thermal management device component comprises compositions that comprise graphene sheets.

2. A thermal management device system comprising a thermal management device component and a computing device component, wherein the thermal management device component comprises at least one heating and/or cooling element that comprises compositions that comprise graphene sheets.

3. The thermal management device system of claim 1, wherein the temperature control element is a heating element.

4. The thermal management device system of claim 1, wherein the temperature control element is a cooling element

5. The thermal management device system of claim 1, wherein the thermal management device component is a heater.

6. The thermal management device system of claim 1, wherein the compositions comprise coatings.

7. The thermal management device system of claim 1, wherein the graphene sheets have a surface area of at least about 300 m²/g.

8. The thermal management device system of claim 1, wherein the computing device component comprises one or more selected from personal computers, smart phones, and tablet computers.

9. The thermal management device system of claim 1, wherein the graphene sheets have a carbon to oxygen molar ratio of at least about 25:1.

10. The thermal management device system of claim 1, wherein the composition further comprises at least one polymeric binder.

11. The thermal management device system of claim 1, wherein the composition is in the form of an ink or coating.

12. A thermal management device system comprising a thermal management device component and a computing device component, wherein the thermal management device component comprises at least one heating and/or cooling element that comprises a coating comprising at least one electrically conductive component.

13. The thermal management device system of claim 12, wherein the electrically conductive component comprises graphene sheets.

14. The thermal management device system of claim 12, wherein the graphene sheets have a surface area of at least about 300 m²/g.

15. The thermal management device system of claim 12, wherein the graphene sheets have a carbon to oxygen molar ratio of at least about 25:1.

16. The thermal management device system of claim 1, wherein the thermal management device component is a medical or therapeutic device.

17. The thermal management device system of claim 1, wherein the thermal management device component is incorporated into an article of apparel.

18. A method of controlling a thermal management device, comprising connecting the thermal management device to a computing device.
